(19) United States
(12) Patent Application Publication
(10) Pub. No.: US 2006/0009732 A1
(43) Pub. Date: Jan. 12, 2006

(54) Method and apparatus for delivering a colonic lavage

(76) Inventor: Michael Hardy, Lawrenceville, GA (US)

Correspondence Address: NEEDLE & ROSENBERG, P.C., SUITE 1000, 999 PEACHTREE STREET, ATLANTA, GA, 30309-3915, US

(21) Appl. No.: 11/170,888

(22) Filed: Jun. 30, 2005

Related U.S. Application Data
(60) Provisional application No. 60/585,533, filed on Jul. 6, 2004.

(51) Int. Cl. A61M 1/00

(52) U.S. Cl. 604/35; 604/27

(57) ABSTRACT

A portable, lightweight bowel evacuation apparatus and method of delivering a colonic lavage is provided. The apparatus includes a liquid bag containing lavage liquid connected to a supply conduit that leads to a nozzle assembly. The apparatus also includes a drain conduit leading from the nozzle assembly to a waste collection. A pulsating member, such as a manual pump or squeeze bulb is provided to generate pulsed waves in the lavage liquid in the patient's colon so as to break up and remove fecal material.

### CROSS-REFERENCE TO RELATED APPLICATIONS

This application claims the benefit of the filing date of U.S. Provisional Patent Application Ser. No. 60/585,533, filed on Jul. 6, 2004, and which is incorporated by reference as if fully set forth in its entirety herein.

### FIELD OF THE INVENTION

This invention generally relates to bowel evacuation systems, and more particularly to apparatus and methods of delivering a colonic lavage and to manual pumps usable in such apparatus and methods.

### BACKGROUND OF THE INVENTION

Many individuals, particularly those who are handicapped or otherwise incapacitated, have substantial difficulty with bowel movements or have no ability at all to voluntarily evacuate their bowels. This condition can lead to serious fecal impactions that require medical attention. Normal treatment may comprise daily enemas and/or manual removal of fecal matter. In extreme cases, a surgical colostomy is required.

Conventional enema treatments often result in inadequate bowel evacuations and their associated problems. Typically, conventional enemas provide for an infusion of water into the individual's colon, but do not provide a means for collecting the wastewater and waste material. During the infusion step, the patient will usually experience cramping in their colon, necessitating stopping the procedure to allow the patient to void. After receiving the infusion of water from an ordinary enema, the patient must quickly mount a bedpan, bedside toilet chair or bathroom toilet in order to dispose of the waste material. Not surprisingly, this step often results in bowel accidents that leave waste material on the bed or floor. As a result, many patients elect to sit on the toilet while the ordinary enema is administered. However, this delivery position can be harmful to the patient, since the colon tends to be vertically aligned and the water pressure pressing sideways on the colon walls can cause the colon to enlarge. Even after contending with these disadvantages, the patient's colon often is not completely cleaned because ordinary enemas usually cannot break up and remove fecal impactions or completely clean the colon in a reasonable amount of time, due in part to the fact that most conventional enemas include water reservoirs that can hold only about one liter of fluid. Accordingly, patients with limited mobility often can have bowel accidents after an enema, leaving them to lie or sit in their own fecal material.

Colonic irrigation devices have been developed but have not proven to be entirely successful. For example, many of the devices are large, bulky, and heavy and suited for use only by medical personnel in the confines of a doctor's office, hospital, or other appropriate facility. Most are not suitable for personal use by a patient in his or her home, and certainly cannot be carried easily on trips away from home. In addition, many conventional devices include pumping and valve mechanisms that tend to deliver lavage liquid to the colon in a harsh manner that can be uncomfortable for the patient and ineffective in removing certain types of fecal impactions. The devices also make it difficult for an operator to determine when the proper amount of lavage liquid has been introduced into the patient, which can result in the patient's colon being insufficiently cleansed. Further, most of these devices include valves and specula that are formed of hard, uncomfortable components and include injection molded plastic parts, which are relatively expensive, especially for single use disposable items. Consequently, there is a need for alternative apparatus and methods for delivering a colonic lavage.

### SUMMARY

The present invention generally encompasses a method and apparatus for delivering a colonic lavage with pulsating action produced by a manual pump. The method generally entails delivering a lavage liquid to a patient's colon, pulsating the lavage liquid within the colon with a manual pump and then draining the liquid from the colon. The apparatus generally includes a nozzle assembly through which lavage liquid can be delivered to the colon of a patient and a manual pump that can pulsate the liquid in the patient's colon.

In one embodiment, the apparatus comprises a lavage liquid reservoir for holding lavage liquid, a supply conduit in fluid communication with the lavage liquid reservoir, and a nozzle assembly with a mouth that is in fluid communication with the supply conduit. In a further aspect, the apparatus comprises a manual pump and a drain conduit, both of which are in fluid communication with the mouth of the nozzle assembly. In a further aspect, the manual pump can be disposed in-line with the supply conduit, such that the lavage liquid flows through the manual pump from the lavage liquid reservoir to the mouth of the nozzle assembly. In a further aspect, a waste receptacle can be provided in fluid communication with the drain conduit so as to receive the contents of the colon when the lavage liquid is drained therefrom.

In a further aspect, a first valve can be disposed between the mouth of the nozzle assembly and the lavage liquid reservoir so as to regulate flow therebetween. Similarly, a third valve can be disposed between the nozzle assembly mouth and the waste receptacle to regulate flow therebetween.

A method of administering a colonic lavage is described in which lavage liquid introduced into the colon is pulsed to provide desired agitation that tends to break up fecal matter and impactions. The method generally includes inserting a nozzle assembly into a rectum of a patient, delivering a liquid, which can be a lavage liquid, a medicant liquid, or a combination of both, through the nozzle assembly to a colon of the patient, pulsating the lavage with a manual pump, and, draining the liquid from the colon.

### BRIEF DESCRIPTION OF THE DRAWINGS

The accompanying drawings, which are incorporated in and constitute a part of this specification, illustrate certain aspects of the instant invention and together with the description, serve to explain, without limitation, the principles of the invention.

FIG. 1 is a schematic illustration of an embodiment of a colonic lavage apparatus of the present invention.

FIG. 2 is a schematic illustration of an alternative embodiment of a colonic lavage apparatus of the present invention.

FIG. 3 is a schematic illustration of an alternative embodiment of a colonic lavage apparatus of the present invention.

FIG. 4 is a schematic illustration of an alternative embodiment of a colonic lavage apparatus of the present invention.

FIG. 5 is a perspective view of an exemplified nozzle member of a nozzle assembly.

FIG. 6 is a cross-sectional view of the nozzle member of FIG. 5.

FIG. 7 is a perspective view of an exemplified nozzle member of a nozzle assembly.

FIG. 8 is a cross-sectional view of the nozzle member of FIG. 7.

FIG. 9 is a partial cross-sectional view of an embodiment of a third valve of the apparatus of the present invention.

### DETAILED DESCRIPTION OF THE INVENTION

The present invention can be understood more readily by reference to the following detailed description, examples, and claims, and their previous and following description.

Before the present compositions, devices, and/or methods are disclosed and described, it is to be understood that this invention is not limited to the specific articles, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. Those skilled in the relevant art will recognize that many changes can be made to the embodiments described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “reservoir” includes aspects having two or more reservoirs unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, the term “colonic lavage” refers to the washing of the colon with one or more infusions of fluid into the colon. The term also encompasses the delivery to and/or removal from the colon of one or more medicants, alone or in conjunction with a washing. It is contemplated that the delivery of medicants encompasses infusion of barium for X-ray procedures, chemotherapy compounds, and other medicinal compounds. While water typically is used as the lavage liquid in the colonic lavage, other liquids, medications, and materials can be delivered using the apparatus of the present invention.

As used herein, a “subject” refers to any living organism having a rectal opening through which lavage fluid can be communicated. For example, the subject can be an animal. In one aspect the animal can be mammalian. Alternatively, the animal can be a warm-blooded animal, such as a human, a farm animal, a domestic animal, or even a laboratory animal. Accordingly, it should be understood that the present invention is not limited to its use in connection with any one particular subject or group of subjects.

Referring now to the drawings, wherein like numerals refer to like features throughout the several views, the figures illustrate features of alternative embodiments of an apparatus for delivering a colonic lavage to a subject. Referring to the figures, an exemplified apparatus 10 is shown that is designed to provide a colonic lavage with the capability of providing manually controlled pulsation to the lavage liquid or medicants through the use of a manual pump 44. In an exemplified embodiment, the apparatus 10 can comprise both a lavage liquid reservoir 20 and a waste receptacle 80 so that the patient need not move in order to remove fecal material from the colon. Further, a colonic lavage or pulsing enema can be delivered to a patient while the patient is on their back or left side, thereby avoiding placing excessive pressure on the walls of the patients colon.

The manual pump 44 can be a conventional in-line squeeze bulb, such as, for example, the squeeze bulb, part V1572X, supplied by Halkey-Roberts, 11600 MLK Jr. Street N., St Petersburg, Fla. In one aspect, the manual pump is disposed in-line with a supply conduit 30. For example, and not meant to be limiting, the manual pump 44 can be formed of a rubber, polyvinyl elastomer or similar material.

The apparatus 10 comprises a nozzle assembly 50 comprising a tubular nozzle member 52 that is sized and shaped for insertion at least partially into a patient's rectum. It will be understood by one skilled in the art that any conventional nozzle member, including, without limitation, conventional rectal tube or rectal catheters, can be used as the nozzle member in the apparatus of the present invention.

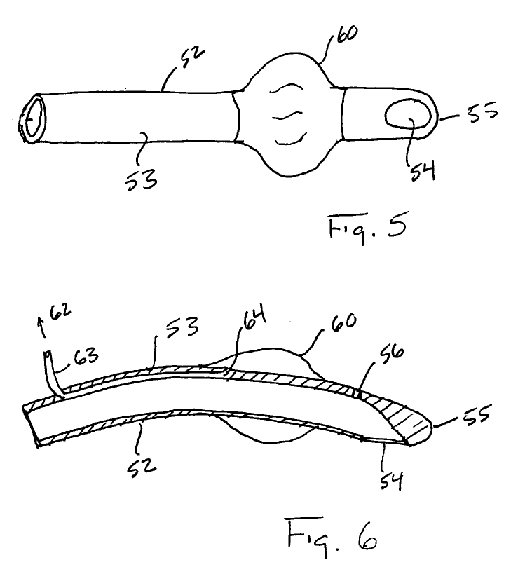

Referring to FIGS. 5-8, two exemplified, and non-limiting, nozzle members 52 are shown. In these examples, each nozzle member 52, which can, for example and not meant to be limiting, be molded from a unitary piece of rubberized material such as silicone or polyvinyl elastomer, has a generally tubular body 53 with a closed distal end in which is defined a mouth 54. Referring to FIGS. 5 and 6, the mouth of the tubular body is defined in a distal end portion of the nozzle member. As shown, the tip portion 55 of the nozzle member can be rounded for ease of insertion. Further, and once again for ease of insertion, the tip portion of the nozzle member can be built up such that it is more rigid that other portions of the tubular body. In this example, the nozzle member 52 can further comprise a bore 56 that extends through the side wall of the nozzle member and is in communication with the interior of the nozzle member. In a further aspect, the bore 56 is spaced from the mouth 54 and can be positioned on the opposite side of the nozzle member relative to the mouth. In a further aspect, the nozzle member 52 can have an elongate lengthwise dimension that has a curved shape.

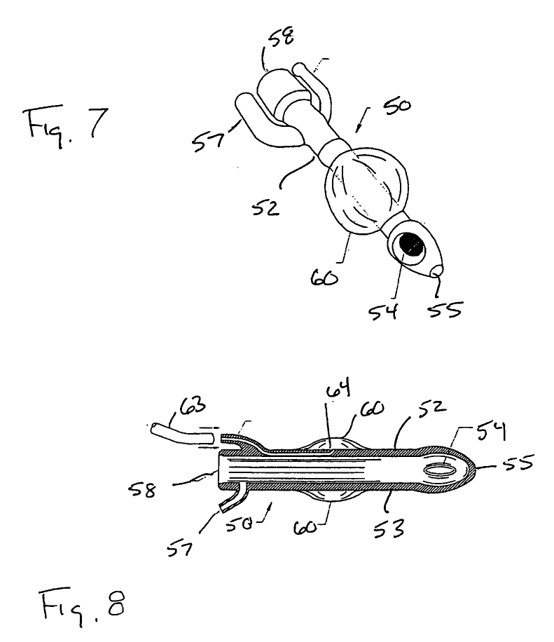

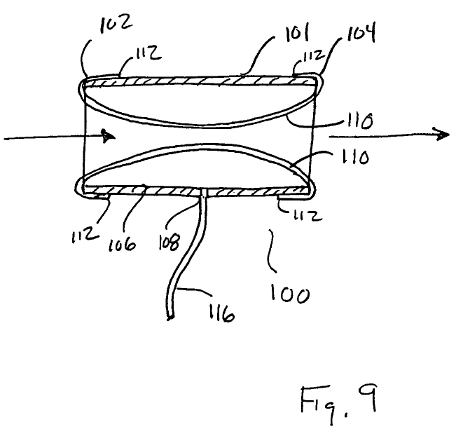

Referring now to FIGS. 7 and 8, in an alternative embodiment, the nozzle member 52 defines an inlet end 57 and an outlet end 58 that are formed in the proximal end portion of the nozzle assembly 50 and are in communication with the mouth 54 of the nozzle assembly.

One will appreciate that the proximal end of the exemplified nozzle member can form both an inlet end 57 and an outlet end 58 if the supply conduit is directly connected to the proximal end of the nozzle member, which is this aspect, forms the nozzle assembly 50. In an alternative embodiment, the nozzle assembly 50 further comprises a three port connector 59. In this aspect, one port of the three port connector is connected to the proximal end of the nozzle member 52 so that the remaining two ports can form the respective inlet and outlet ends 57, 58 of the nozzle assembly.

The nozzle assembly 50 can also comprise a selectively inflatable cuff 60 that is formed on a portion of the exterior surface of the tubular body portion of the nozzle member 52 and is selectively inflated and deflated by use of a cuff pump 62. In one example, the inflatable cuff 60 is positioned circumferentially about the exterior surface of the nozzle member in between the inlet end and the mouth of the nozzle assembly 50. The cuff pump 62 is in flow communication with an air inlet 64 on the nozzle member 52 that is positioned under the inflatable cuff 60. For example, the cuff pump 62 can be a polyvinyl squeeze bulb to which is attached an inline cutoff valve or with which a clamp 65 is provided attached to the tube 63 leading to the air inlet 64 of the nozzle assembly 50. The tube leading from the cuff pump 62 to the air inlet 64 can comprise flexible tubing made from vinyl or other polymeric material. Alternatively, it is contemplated that at least a portion of the tube can be integrally formed in a portion of the sidewall of the nozzle member. An exemplary inflatable cuff 60 is the silicone cuff, material # V825A-028, supplied by Vesta, Inc., of Franklin, Wis. A small hole is created in the bottom layer of the cuff to allow air to enter the cuff from the air inlet 64 on the side of the nozzle member 52. An adhesive, disposed between the inflatable cuff 60 and the nozzle member 52, prevents air leakage.

In one aspect of the apparatus of the present invention, an end of the supply conduit 30 is secured to the inlet end 57 of the nozzle assembly 50. In one embodiment, the supply conduit 30 comprises a first section 32 and a second section 33. The first section 32 of the supply conduit has a proximal end that is in fluid communication with the lavage liquid reservoir 20 and a distal end. The second section 33 of the supply conduit has a distal end that is secured to the inlet end 57 of the nozzle assembly and is in fluid communication with the mouth 54 of the nozzle assembly and a proximal end. The distal end of the first section of the supply conduit is adapted to releasably connect, via conventional connectors 34, to the proximal end of the second section of the supply conduit. The sections of the supply conduit 30 can be formed from conventional medical tubing and can, for example, be formed of vinyl or other polymeric tubing.

In one example, the manual pump 44 is disposed in-line with the second section 33 of the supply conduit 30. In one aspect, conventional and complementary connectors 42 interconnect the manual pump 44 with the supply conduit. The proximal end of the first section 32 of the supply conduit 30 is connected to a connector 24 on the lavage liquid reservoir 20, which is shown as a polymeric bag, but also can include other suitable containers.

For example, and not meant to be limiting, the lavage liquid reservoir 20 can be formed of a polymeric material, such as polyethylene, polypropylene, or rubber. In one embodiment, the lavage liquid reservoir is a single-use 3 mil polyethylene bag. The lavage liquid reservoir 20 has a handle 22 or other suspension member that can be used to hang the reservoir from a stand so as to elevate the reservoir above the patient to allow gravity feed of liquid to the patient. Typically, the liquid lavage liquid reservoir 20 is placed approximately 2′ over a bed or other surface upon which the patient lies. The lavage liquid reservoir can also comprise a flap valve 26 in communication with an interior volume of the reservoir such that the reservoir can be selectively filed with the desired liquid. Alternatively, the conduit leading into the lavage liquid reservoir 20 can be used as a funnel to fill the interior volume of the reservoir. While water is usually the liquid chosen to be delivered, it is contemplated that other liquids, medications, medicants, and the like may be delivered using the apparatus of the present invention.

A first valve 36 is operably connected to the supply conduit 30 and is adapted for selective regulation of fluid flow between the lavage liquid reservoir 20 and the mouth 54 of the nozzle assembly. Preferably, the first valve 36 is positioned between the manual pump 44, which is positioned in-line with the supply conduit, and the lavage liquid reservoir 20. In one aspect, the first valve 36 can be operatively connected to the first section 32 of the supply conduit 30. The first valve can be a clamp valve, such as, for example the slide clamp, part no. 11034, or the pinch clamp, part no. 13997, supplied by Qosina of Edgewood, N.Y. However, one skilled in the art would appreciate that alternative valve structures are contemplated, such as, for example, a valve disposed in-line with the supply conduit 30.

In another aspect, the first valve 36 is operatively connected to the second section of the supply conduit. In this aspect, the apparatus can comprise a second valve 38 that is operatively connected to the first section of the supply conduit and that is adapted for selective regulation of fluid flow between the lavage liquid reservoir and the distal end of the first section of the supply conduit. The second valve 38 is shown as a clamp valve, such as the exemplified slide or pinch clamp, but also can include alternative valve structures.

The first valve 36 is positioned between the manual pump 44 and the lavage liquid reservoir 20 so that liquid that is delivered to the patient's colon through the nozzle assembly 50 can flow back to the manual pump 44, but not to the lavage liquid reservoir 20. In an alternative embodiment, a check valve, not shown, can be disposed in-line with the supply conduit 30 or the manual pump 44. The check valve can allow the manual pump 44 to be used to pump liquid from the lavage liquid reservoir 20 into the nozzle assembly 50, thereby providing an alternative to gravity feed. Once the desired amount of liquid is delivered to the patient's colon, the check valve can be closed and the manual pump 44 operated as set forth below.

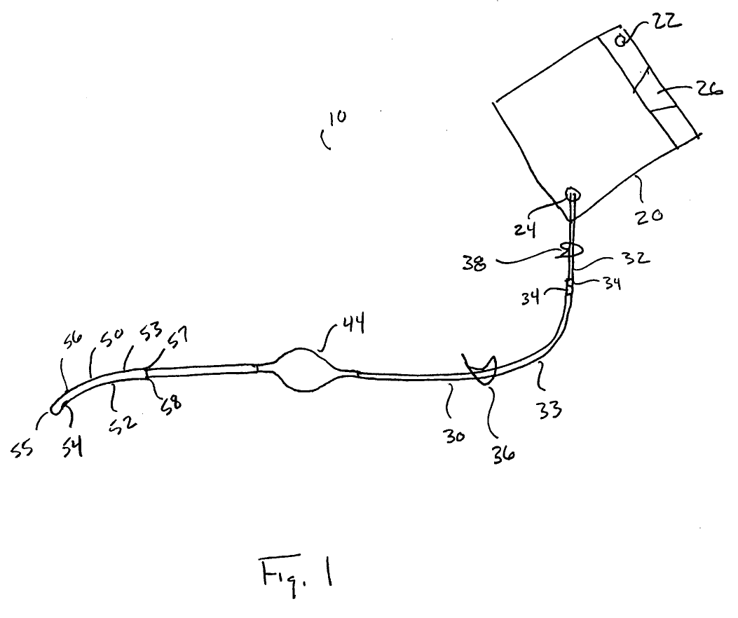

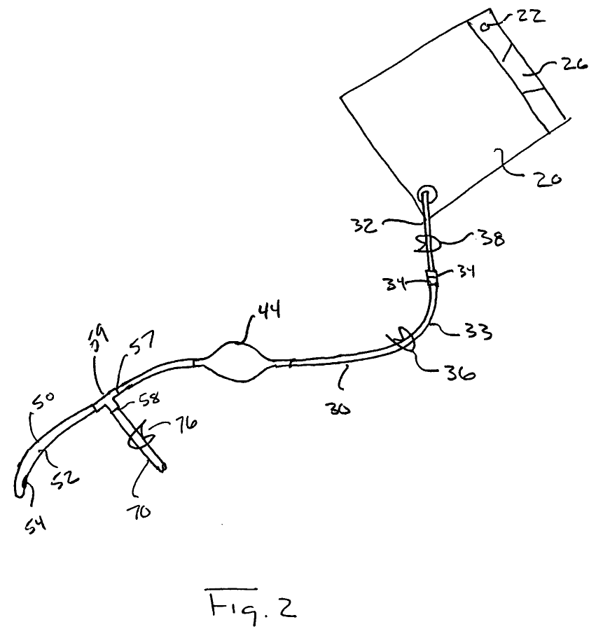

The apparatus of the present invention can also comprise a drain conduit 70 that is connected to the outlet end 58 of nozzle assembly 50. The drain conduit 70 is in flow communication with the mouth 54 of the nozzle assembly 50 and is adapted to allow material washed from the colon to be drained through the nozzle assembly. In a further aspect, the drain conduit 70 is in flow communication with the waste receptacle 80, which is shown in FIG. 1 as a polymeric bag, such as those supplied by Ascent Medical Corporation of Niles, Ill. In one aspect, the drain conduit can be formed of two sections that can be releasable connected by conventional releasable connectors 82 that allow the waste receptacle to be releasably connected to the apparatus of the present invention.

In one aspect, an exemplified waste receptacle 80 can have an upper surface 83, a top edge 84 and an opposed bottom edge 85. A distal end portion of one of the sections of the drain conduit 70 can be mounted to the upper surface of the receptacle such that the drain conduit is in fluid communication with the interior of the receptacle. In one aspect, the section of the drain conduit is mounted to the upper surface 83 of the waste receptacle proximate the top edge 84 of the waste receptacle. The waste receptacle 80 can further comprise a pair of opposed handles 86 that are defined near the corners of the waste receptacle proximate the top edge of the waste receptacle. In one aspect, the opposed pair of handles 86 are positioned closer to the top edge 84 of the waste receptacle than the section of the drain conduit 70. In use, when the section of the drain conduit is disconnected from the rest of the apparatus, the section of the drain conduit 70 can be folded down onto the upper surface 83 of the waste receptacle and the opposing pair of handles 86 can be brought together such that the section of the drain conduit cannot fall away from the upper surface 83 of the receptacle. In a further aspect, the waste receptacle 80 can define a bottom handle 87 near the bottom edge 85 of the waste receptacle. The bottom handle allows for easier handling of the waste receptacle.

In a further aspect, the apparatus of the present invention comprises a third valve 76 that is operably connected to the drain conduit 70. The third valve is adapted to selectively regulate the flow of liquid between the nozzle assembly 50 and the waste receptacle 80. One skilled in the art will appreciate that when the third valve 76 is in its open configuration, waste liquid is free to flow through the outlet end 58 of the nozzle assembly 50, through the drain conduit 70, and thence to the waste receptacle 80. Conversely, when the third valve 76 is closed, the drain conduit 70 is closed and material cannot flow from the nozzle assembly 50 to the waste receptacle 80.

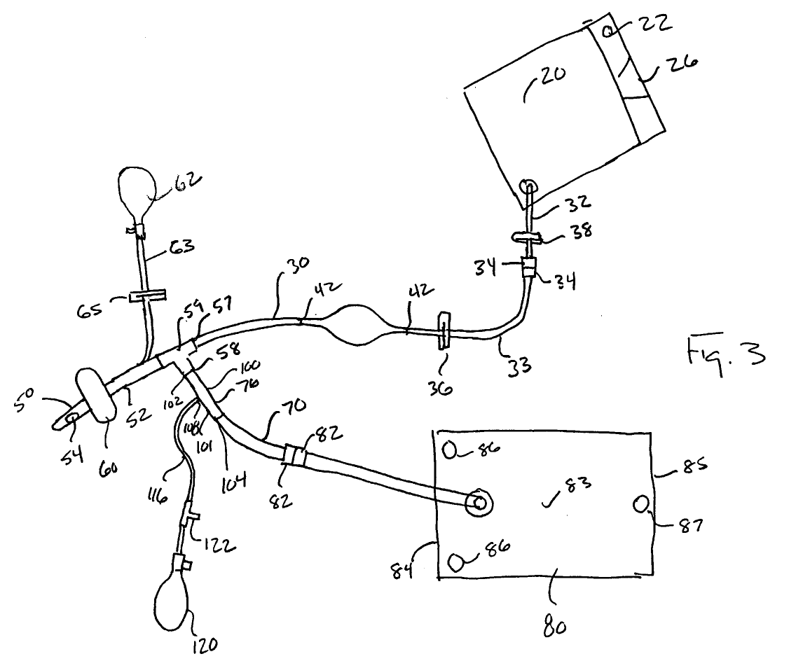

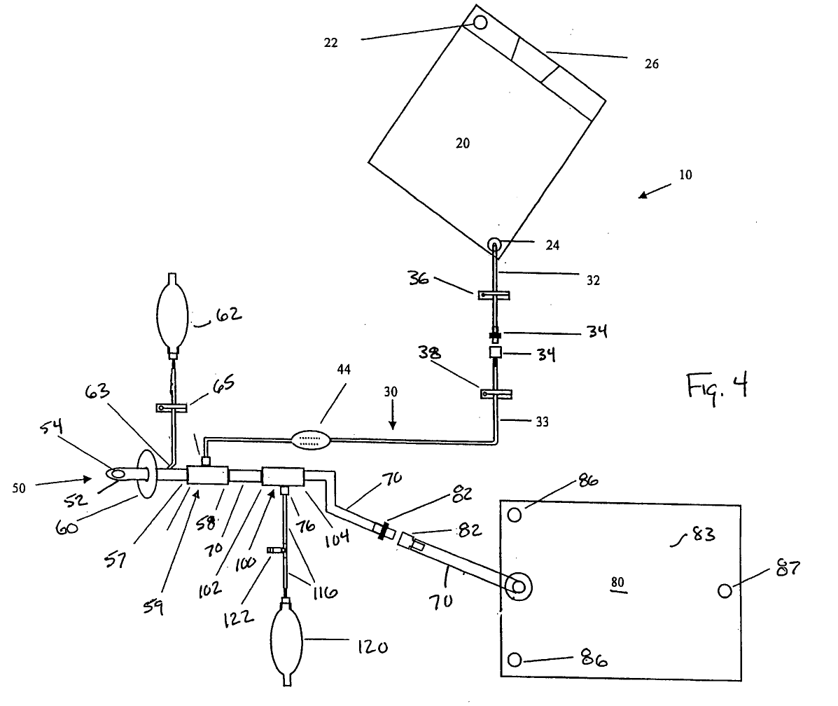

In one example, the third valve 76 can comprise a clamp valve, such as a pinch or slide clamp, but also can include alternative valve structures. For example, and as shown in FIGS. 3, 4 and 9, the third valve 76 can comprise a pressure regulation valve 100 having a sleeve member 101 that has a first end 102 in communication with the mouth 54 of the nozzle assembly and a second end 104 in communication with the waste receptacle 80. The sleeve member 101 has a pliable lining 110, such as, for example, a silicone lining, that has edges 112 that are connected to and extend circumferentially about a portion of an interior surface 106 of the sleeve member. In one example, the edges 112 of the lining 110 are sealed over the outside edge of the respective first and second ends 102, 104 of the sleeve member. The lining 110 is selectively movable about and between a first, inflated position, in which the pliable lining is expanded to occlude the sleeve member 101 such that fluid cannot pass through the sleeve member from the first end 102 to the second end 104, and a second, open position, in which fluid is allowed to pass from the first end 102 to the second end 104 of the sleeve member 101.

The sleeve member 101 defines a port 108 in fluid communication with an interior volume of the pliable lining 110. A fluid line 116 is connected to the port and is in communication with a pump 120, such as, for example, an inflation squeeze bulb or the like. The pump 120 is adapted to allow an operator to selectively increase or release pressure in the connected fluid line. An exemplary pump could be a conventional pump of the type used in conventional, manual blood pressure cuffs.

For safety and comfort, the apparatus 10 of the present invention can also comprise a selectable pressure release valve 122 that is mounted in-line with the fluid line. Exemplarily, the pressure release valve 122 is positioned intermediate the port 108 in the sleeve member and the pump 120 and is adapted to allow the lining 110 to move from the first position to the second, open position at a predetermined pressure such that pressures in excess of the predetermined pressure are not created inside of the colon of the patient. Exemplary predetermined pressures can comprise about 1.5 psi to about 4.5 psi; alternatively about 2.0 psi to about 4.0 psi; or about 2.5 psi to about 3.5 psi.

In one aspect, the conduits/lines used in the apparatus 10 are formed of collapsible polymeric material, such as polyethylene, polypropylene, or rubber, so that the conduits/lines can be selectively closed upon the application of clamp valves.

The apparatus 10 of the present invention is constructed such that the nozzle assembly 50, supply conduit 30, manual pump 44, lavage liquid reservoir 20, drain conduit 70 and waste receptacle 80 are disposable and can be removed or disconnected from the other elements of the system and replaced without disturbing or affecting the other elements of the system.

In practice, the lavage liquid reservoir 20 is filled with body temperature water or other suitable liquid and then is either suspended in an elevated position above the patient or placed on the bed or chair adjacent the patient. In one example that is not meant to be limiting, the lavage liquid reservoir 20 can hold approximately 1.0 gallons of liquid, although capacities are contemplated. The lavage liquid reservoir 20 then is placed on the bed/surface, or hung from a hook/IV stand for gravity feed. The height of the lavage liquid reservoir 20 is such that the head or pressure level of the liquid is sufficient to deliver the liquid through the supply conduit 30, manual pump 44 and nozzle assembly 50 into the patient's colon. As an alternative to gravity feed, the lavage liquid reservoir 20 can be placed on the bed or other surface and gently compressed with hand pressure to force the liquid through the apparatus and into the patient's colon. In one example, the first and second sections of the supply conduit are connected such that the manual pump 44 is in communication with the liquid reservoir.

The tip of the nozzle member 52 of the nozzle assembly 50 is lubricated with a lubrication jelly and inserted into the rectum of the patient. Alternatively, the nozzle assembly 50 is inserted into a colostomy site in the patient.

The nozzle assembly 50 is inserted into the patient's rectum up to the collar or anal ring, and the cuff 60, if used, is inflated to an extent sufficient to prevent the nozzle assembly 50 from accidentally slipping out of the rectum during the bowel evacuation procedure or material leaking during the procedure. The cuff pump 62 is repeatedly compressed to inflate the inflatable cuff 60 sufficiently to fill the rectal vault of the patient. Once inflated to the desired extent, the air line is closed off with a suitable closure to maintain the cuff 60 in the inflated condition.

The waste receptacle 80 is placed below the patient, such as on the floor, so that when the liquid is drained it can flow by gravity from the patient into the receptacle, thereby avoiding suction from being generated and which could possibly harm the patient.

The third valve 76 is closed to prevent liquid from draining from the nozzle assembly 50. The first valve 36, and, if used, the second valve 38, are then opened to allow liquid to flow from the reservoir 20 through the supply conduit 30 into the nozzle assembly 50, through the mouth 54 and into the patient's colon. When the desired amount of lavage liquid is infused, the first valve 36 is closed.

With the first valve 36 closed, either the patient or an assistant begins to compress the manual pump 44 to pressurize the liquid, thereby causing a pulse of liquid to enter the patient's colon and return to refill the squeeze bulb with liquid. Repeatedly squeezing the manual pump 44 pulsates the liquid, which undulates in waves into and out of the patient, thereby tending to break up the fecal material in the patient's colon. It has also been found through clinical trials that pulsating the lavage liquid triggers autonomic peristaltic action in the patient's colon, which further loosens and breaks up impacted waste material.

After pulsating the liquid, the third valve 76 is opened, allowing the waste liquid and stool to exit the colon through the mouth 54 of the nozzle assembly 50 and drain into the drain conduit 70 and the waste receptacle 80. After the waste liquid drains into the waste receptacle 80, the process can be repeated. The process is repeated until it is determined that sufficient stool has been removed from the patient. The waste receptacle 80 can be detached from the rest of the apparatus 100, such as by disconnecting the complementary connectors 82, and carried to a toilet for emptying. Handles 86, 87 can be provided to facilitate the transport of the waste receptacle 80 or for suspending it in a convenient location. The process generally provides for the repeated infusion of pulsing liquid into the patient's colon leading to rehydration and break-up of the stool material into particles small enough to exit the colon through the nozzle assembly 50 and flow into the waste liquid bag 80.

As noted above, the invention has been described herein in terms of particular embodiments and methodologies and it is contemplated that various modifications might well be made to the illustrated embodiments within the scope of the invention. For example, while silicone or a silicone compound has been illustrated as one material from which the nozzle assembly is molded, other appropriate materials can be substituted. Further, the size and relative dimensions of the various components of the nozzle assembly, reservoirs and lines can be varied depending upon the intended use. For example, the manual pump or squeeze bulb can be differently shaped or sized providing a more pronounced pulsed action. Also, the manual pump can be positioned inline with the drain conduit of the assembly or in flow communication with the nozzle assembly through a line that is separated from both the supply conduit and the drain conduit. Finally, the apparatus for delivering a colonic lavage and its various components as described herein may well be constructed with a wide variety of shapes and configurations that could differ from the illustrated embodiments. These and other additions, deletions, and modifications might well be made to the disclosed embodiments by those of skill in the art without departing from the spirit and scope of the invention as set forth in the claims.

What is claimed is:

1. An apparatus for delivering a colonic lavage comprising:
a lavage liquid reservoir;
a supply conduit in fluid communication with the lavage liquid reservoir;
a nozzle assembly having a mouth formed therein, wherein the supply conduit is in fluid communication with the mouth of the nozzle assembly; and
a manual pump in communication with the supply conduit.

2. The apparatus of claim 1, wherein the manual pump is disposed in-line with the supply conduit.

3. The apparatus of claim 1, further comprising a first valve that is adapted for selective regulation of fluid flow between the lavage liquid reservoir and the mouth of the nozzle assembly.

4. The apparatus of claim 3, wherein the manual pump is in-line with the supply conduit.

5. The apparatus of claim 4, wherein the first valve is positioned between the manual pump and the lavage liquid reservoir.

6. The apparatus of claim 5, wherein the first valve is a clamp valve.

7. The apparatus of claim 5, wherein the supply conduit has a first section and a second section, wherein the first section of the supply conduit has a proximal end in fluid communication with the lavage liquid reservoir and a distal end, wherein the second section of the supply conduit has a proximal end and a distal end that is in fluid communication with the mouth of the nozzle assembly, and wherein the distal end of the first section of the supply conduit is adapted to releasably connect to the proximal end of the second section of the supply conduit.

8. The apparatus of claim 7, wherein the manual pump is disposed in-line with the second section of the supply conduit.

9. The apparatus of claim 7, wherein the first valve is mounted to the first section of the supply conduit.

10. The apparatus of claim 7, wherein the first valve is mounted to the second section of the supply conduit, and further comprising a second valve mounted to the first section of the supply conduit that is adapted for selective regulation of fluid flow between the lavage liquid reservoir and the distal end of the first section of the supply conduit.

11. The apparatus of claim 10, wherein the second valve is a clamp valve.

12. The apparatus of claim 1, further comprising a drain conduit in fluid communication with the mouth of the nozzle assembly.

13. The apparatus of claim 12, further comprising a waste receptacle in fluid communication with the drain conduit.

14. The apparatus of claims 5 or 10, further comprising a third valve that is adapted for selective regulation of flow between the mouth of the nozzle assembly and the waste receptacle.

15. The apparatus of claim 14, wherein the third valve is a clamp valve.

16. The apparatus of claim 14, wherein the third valve comprises:
a sleeve member having a first end in communication with the mouth of the nozzle assembly and a second end in communication with the waste receptacle, wherein the sleeve member has a pliable lining having edges that are connected to and extend circumferentially about a portion of an interior surface of the sleeve member, the pliable lining selectively movable about and between a first, inflated position, in which the pliable lining is expanded to occlude the sleeve member such that fluid cannot pass from the first end to the second end of the sleeve member, and a second, open position, in which fluid is allowed to pass from the first end to the second end of the sleeve member, and wherein the sleeve member defines a port in fluid communication with an interior volume of the pliable lining; and
a pump in fluid communication with the port of the sleeve member.

17. The apparatus of claim 16, further comprising:
a fluid line extending therebetween the pump and the port of the sleeve member; and
a selectable pressure relief valve in communication with the fluid line that is adapted to allow the pliable member to move from the first, inflated position to the second, open position.

18. The apparatus of claim 13, wherein the waste receptacle is releasably connected to the drain conduit.

19. The apparatus of claim 1, further comprising a handle attached to the lavage liquid reservoir.

20. The apparatus of claim 1, further comprising an inflatable cuff formed on a portion of the exterior surface of the nozzle assembly.

21. The apparatus of claim 20, wherein the nozzle assembly has an inlet end that is spaced from the mouth of the nozzle assembly, and wherein the inflatable cuff is positioned circumferentially about the exterior surface of the nozzle assembly in between the inlet end and the mouth of the nozzle assembly.

22. The apparatus of claim 20, further comprising a cuff pump in fluid communication with the inflatable cuff.

23. The apparatus of claim 1, wherein the lavage liquid reservoir is releasably connected to the supply conduit.

24. The apparatus of claim 1, wherein the nozzle assembly comprises an inlet end connected to one end of the supply conduit and an outlet end connected to an end of the drain conduit.

25. The apparatus of claim 1, wherein the lavage liquid reservoir can comprise a medicant reservoir.

26. An apparatus for delivering a colonic lavage comprising:
a liquid reservoir;
a supply conduit in fluid communication with the lavage liquid reservoir;
a nozzle assembly having a mouth formed therein and an inlet end spaced from and in fluid communication with the mouth, wherein the supply conduit is in fluid communication with the inlet end of the nozzle assembly;
a manual pump positioned in-line with the supply conduit and in fluid communication with the mouth of the nozzle assembly; and
a drain conduit in fluid communication with the mouth

27. The apparatus of claim 26, further comprising a waste receptacle in fluid communication with the drain conduit.

28. The apparatus of claim 27, wherein the liquid reservoir defines an interior volume, and wherein the interior volume of the liquid reservoir is filed with a fluid selected from the group consisting of a lavage liquid, a medicant liquid, or a combination of both.

29. The apparatus of claim 28, wherein the liquid reservoir comprises a refill valve in fluid communication with the interior volume of the liquid reservoir.

30. The apparatus of claim 26, wherein the supply conduit comprises a first section releasably connected to a second section, the first section in fluid communication with the lavage liquid reservoir and the second section in fluid communication with the mouth of the nozzle assembly.

31. The apparatus of claim 30, wherein the manual pump is disposed in-line with the second section of the supply conduit.

33. The apparatus of claim 30, further comprising a first valve disposed between the manual pump and the lavage liquid reservoir.

34. The apparatus of claim 33, wherein the first valve is mounted to the second section of the supply conduit.

35. The apparatus of claims 34, further comprising a second valve mounted to the first section of the supply conduit that is adapted for selective regulation of fluid flow between the lavage liquid reservoir and a distal end of the first section of the supply conduit.

36. The apparatus of claims 33 or 35, further comprising a third valve that is adapted for selective regulation of flow between the mouth of the nozzle assembly and the waste receptacle.

37. The apparatus of claim 26, further comprising:
an inflatable cuff attached to the nozzle assembly; and
a cuff pump in fluid communication with the inflatable cuff.

38. A method for delivering a colonic lavage comprising:
a) inserting a portion of a nozzle assembly into a patient;
b) delivering a lavage liquid through the nozzle assembly to a colon of the patient;
c) pulsating the lavage liquid with a manual pump; and,
d) draining the lavage liquid from the colon.

39. The method of claim 38, further comprising delivering a medicant through the nozzle assembly to the colon of the patient.

40. The method of claim 39, further comprising pulsating the medicant in the colon of the patient with a manual pump.

41. The method of claim 39, further comprising draining the medicant through the nozzle assembly from the colon of the patient.

42. The method of claim 39, further comprising delivering a second charge of lavage liquid through the nozzle assembly to the colon of the patient.

43. The method of claim 42, further comprising draining the second charge of lavage liquid through the nozzle assembly from the colon of the patient.

44. The method of claim 38, further comprising closing a first valve to maintain the lavage liquid in the colon of the patient.

45. The method of claim 44, further comprising opening a third valve to drain the lavage liquid from the colon of the patient.

46. The method of claim 38, further comprising inflating a cuff to secure the nozzle assembly in the rectum of the patient.

47. The method of claim 38, further comprising draining the lavage liquid into a waste receptacle in fluid communication with the nozzle assembly.

48. The method of claim 38, wherein the step of inserting the nozzle assembly comprises inserting at least a portion of a nozzle member of the nozzle assembly therein a rectum of the patient.

49. The method of claim 38, wherein the step of inserting the nozzle assembly comprises inserting at least a portion of a nozzle member of the nozzle assembly therein a colostomy site of the patient.
